(19) United States
(12) Patent Application Publication
(10) Pub. No.: US 2024/0083973 A1
(43) Pub. Date: Mar. 14, 2024

(54) T CELLS MODIFIED TO EXPRESS MUTATED CXCR4 OR PARTIALLY DELETED AND USES THEREOF

(71) Applicants: ECOLE NORMALE SUPÉRIEURE DE LYON, Lyon (FR); UNIVERSITÉ CLAUDE BERNARD - LYON 1, Villeurbanne (FR); UNIVERSITÉ PARIS-SACLAY, Saint Aubin (FR)

(72) Inventors: Karl BALABANIAN, Clamart (FR); Jacqueline MARVEL, Lyon (FR); Mélanie WENCKER, Lyon-Cedex 07 (FR); Sophia DJEBALI, Lyon (FR)

(21) Appl. No.: 17/766,648

(22) PCT Filed: Oct. 8, 2020
(86) PCT No.: PCT/EP2020/078283

§ 371 Date: Apr. 5, 2022

(30) Foreign Application Priority Data
Oct. 9, 2019 (EP) 19306319.5

(51) Int. Cl. C07K 14/715; A61K 39/00; C12N 5/0783

(52) U.S. Cl. CPC C07K 14/7158; A61K 39/4611; A61K 39/4635; C12N 5/0638; C12N 2510/00

(57) ABSTRACT

The present invention relates to modified T cells, and in particular to CD8 T cells, for use in therapy. The current inventors investigates the effect of CXCR4^{Whim} mutation on CD8 effector and memory responses. By analysing the lymphoid organs, including the BM compartment, in a mouse model of Whim syndrome and in mice where only CD8 T-cells carry the mutation, this study shows that CXCR4^{Whim} mutation only partially affect CD8 primary responses. By contrast, CXCR4^{Whim} mutation considerably improve the long-term maintenance and magnitude of CD8 memory responses by increasing the pool size of Antigen specific CD8 T-cells, first in the BM and then in other lymphoid organs, bringing new insight into the current discrepancy regarding the role of the BM in the maintenance CD8 memory cells. In particular, the present invention relates to T cell, and more particular to CD8 T cell, characterized in that it expresses CXCR4^{Whim} mutation or a CXCR4 with the deletion of the C-terminal domain between and 20 amino acid residues.

### FIELD OF THE INVENTION

The present invention relates to T cell characterized in that it expresses CXCR4^{Whim} mutation or a CXCR4 with the deletion of the C-terminal domain between 10 and 20 amino acid residues and their use for the treatment and prevention of infectious disorders and cancers.

### BACKGROUND OF THE INVENTION

Chemokines are small soluble factors that regulate cells positioning and migration through binding to their respective receptor expressed on cells surface. They play crucial role during immune responses, participating in spatial and temporal establishment of immune cells to allow optimal immune responses¹. An interesting example on how disruption of chemokine/chemokine receptor can affect immune responses is illustrated by Whim (Warts, Hypoglobulinemia, Infection and Myelokathexis) syndrome, a rare immunodeficiency disease that is characterized by recurrent bacterial infection and increased susceptibility to HPV infection and HPV-induced carcinogenesis².

Indeed, at the molecular level Whim syndrome is caused by an autosomal dominant mutation in gene encoding CXCR4 chemokine receptor³⁻⁷. CXCR4 is a G-protein coupled receptor that is expressed on a variety of haematopoietic cell types, including neutrophils, B and T-cells¹ and regulates cellular migration in a very timely manner. CXCR4 main ligand is CXCL12 (also called SDF-1), a homeostatic chemokine that is highly secreted in spleen, Lymph Nodes (LN) and bone marrow (BM)⁸. Importantly, CXCL12 is also secreted at inflammation sites⁹, allowing the recruitment of several players of immune responses. In the absence of its ligand, CXCR4 is expressed at the surface of cells in an inactive form. Engagement of CXCL12 leads to receptor activation and downstream signalling but it is ultimately desensitized, in a process that is mediated either by receptor internalization or return into inactive form¹⁰. Several CXCR4 mutations have been described in Whim patients³⁻⁷, most of all affecting the C-terminal part of CXCR4 receptor that is involved in receptor desensitization, thus impairing internalization and/or receptor inactivation. Consequently, Whim mutations act as gain-of-function mutations, leading to increased responsiveness to CXCR4 main ligand, CXCL12.

One major difficulty regarding Whim syndrome is the accessibility to patients samples, since it is a very rare disease and patients show deep immune defect that prevent regular sampling. Moreover, Whim mutations affect migration of cells through several tissues (including lymph nodes, peripheral sites, and bone marrow) to which access remains hardly possible. To circumvent this issue, experimental mice models have been established, that carry whim mutation and nicely recapitulate the human syndrome⁶. Altogether, rare patients' samples and mice models allowed important understanding of immune defect at the origin of Whim associated pathology. Thus, CXCR4 impaired responsiveness has several consequences: increased CXCL12 responsiveness retain neutrophils in the BM leading to important neutropenia and myelokathexis¹¹. Additionally, T and B-cells lymphopenia is observed in Whim patients as well as a decrease in serum level of Immunoglobulin, probably due to defective B-cells development and improper trafficking of B cells through BM⁶. Consequently, Whim patients show normal B cells response during primary challenges but impaired secondary responses⁷, probably due to abnormal isotype switching, defective maintenance of antibody producing cells¹² and impaired differentiation of plasma cells^{13 14}. Considering the role of B cells and neutrophils in responses against extra-cellular bacterial infection, those observations highly correlate with patients' high susceptibility to infection.

Surprisingly, little or no study attempted to decipher the effect of CXCR4^{Whim} mutations on CD8 T-cells responses. CD8 T-cells are important players of anti-viral and anti-tumor responses, with migration and motility being central to their optimal functions. For example, naïve CD8 T-cells rely on CCR7 and SP receptor to circulate through secondary lymphoid organs (SLO) and contact Antigen-presenting cells (APC) during infection¹⁵. Once a primary response occurred, memory CD8 T-cells survive and acquire a novel set of chemokine and chemokines receptors that allow them to scan peripheral tissue and rapidly access the site of infection/tumor in case of secondary encounter with the same antigen¹⁶. The past decade also highlighted the importance of resident memory CD8 T-cells in creating an alert state within tissue that is required for optimal recruitment and progression of immune responses¹⁷. CXCR4 is expressed on CD8 T-cells and participate in the recruitment of naïve cells within the LN¹⁵, effector cells to inflamed tissue⁹ and memory cells in the BM^{18, 19}. In line with this, several studies have pointed out a role for the BM as a site of maintenance for CD8 memory T-cells¹⁹⁻²¹, although discrepancies remain on whether the BM provide a privileged site for proliferation and/or survival of memory CD8 T-cells^{22,23}.

### SUMMARY OF THE INVENTION

The present invention relates to T cells and uses thereof. More particularly, the present invention relates to T cell characterized in that it expresses CXCR4^{Whim} mutation or a CXCR4 with the deletion in the C-terminal domain between 10 and 20 amino acid residues and their use for the treatment and prevention of infectious disease and cancers. In particular, the invention is defined by the claims.

### DETAILED DESCRIPTION OF THE INVENTION

The current inventors investigated the effect of CXCR4^{Whim} mutation on CD8 effector and memory responses. By analysing the BM compartment in a mouse model of Whim syndrome (Whim mice 6) and in mice where only CD8 T-cells carry the mutation, this study shows that CXCR4^{Whim} mutation only partially affects CD8 primary responses, suggesting that Whim patients can mount somewhat efficient anti-viral (or anti tumoral) CD8 responses. By contrast, CXCR4^{whim} mutation in CD8 T-cells considerably improve the long-term maintenance and magnitude of CD8 memory responses by increasing the pool size of Antigen specific CD8 T-cells in the BM, bringing new insight into the current discrepancy regarding the role of the BM in the maintenance CD8 memory cells.

Altogether, their findings thus identified that expression of CXCR4^{Whim} on CD8 T cells considerably improves the long-term maintenance and magnitude of CD8 memory responses by increasing the pool size of antigen specific CD8 T-cells and thus could constitute a valuable new tool in therapy to increase CD8 T-cells numbers and activities against a specific antigen. Moreover, in cell therapy and in adoptive cell therapy, especially with CarT cells, an issue to be solved concern the activity of T cells, and in particular CD8 T cells, in the long term, and the present invention represents a solution to this problem.

T Cells of the Invention

Accordingly, a first aspect of the present invention relates to a T cell characterized in that it expresses a CXCR4^{Whim} mutation or a CXCR4 with the deletion in the C-terminal domain of between 10 and 20 amino acid residues.

Thus, the present invention relates to T cell characterized in that it expresses a CXCR4^{Whim} mutation or a CXCR4 with the deletion in the C-terminal domain of between 10 and 20 amino acid residues for use in therapy.

This permits to increase the long-term maintenance and magnitude of T cell response, and in particular CD8 memory responses, and also the activity of T-cells by increasing the pool size of Antigen specific T-cells.

The term “T cells” (also called “T lymphocytes”) represent an important component of the immune system that plays a central role in cell-mediated immunity. T cells are known as conventional lymphocytes as they recognize a specific antigen with their TCR (T Cell Receptor for the antigen) with presentation or restriction by molecules of the complex major histocompatibility. There are several subsets of T cells each having a distinct function such as CD8+ T cells, CD4+ T cells, regulatory T-cells . . . .

In some embodiment, the T cell is CD8+ T cell, CD4+ T cell or Gamma delta (γδ) T cell.

The term “CD8+ T cells” (also called Cytotoxic T cells or TC cells, CTLs, T-killer cells or killer T cells) is used to describe T lymphocytes, which express the CD8 glycoprotein at their surface and when activated by host cells presenting specific antigens (APCs) via MHC I, are able to destroy infected cells and tumor cells, presenting the same antigens on their surface. Naïve CD8+ T cells have numerous acknowledged biomarkers known in the art. These include in human CD45RA+CCR7+HLA-DR-CD8+ and the TCR chain is formed of an alpha chain (α) and a beta chain (β).

The term “CD4+ T cells” (also called T4 cells, T helper cells, Tₕ cells) is used to describe T lymphocytes, which express the CD4 glycoprotein at their surface, and play an important role in the immune system, particularly in the adaptive immune system. As their name suggests, they “help” the activity of other immune cells by releasing cytokines, small protein mediators that alter the behavior of target cells that express receptors for those cytokines. CD4 is a co-receptor of the T cell receptor (TCR) and assists the latter in communicating with antigen-presenting cells. The TCR complex and CD4 bind to distinct regions of the antigen-presenting MHC class II molecule.

The term “Gamma delta (γδ) T cell” is used to describe T lymphocytes that have a distinctive T-cell receptor (TCR) on their surface. While CD4+ and CD8+ T-cells are αβ (alpha beta) T cells with TCR composed of two glycoprotein chains called α (alpha) and β (beta) TCR chains, gamma delta (γδ) T cells have a TCR that is made up of one γ (gamma) chain and one δ (delta) chain. They mainly reside in non-lymphoid tissues and can be divided in several subsets that can respond rapidly to innate-type of signals (such as cytokines and stress-associated molecules) and/or antigens recognized by their TCR in a MHC-independent way. They play important roles during immune responses against viral and bacterial infection, and cancer.

In some embodiment, the T cell is CD8+ T cell.

Thus, the present invention relates to a CD8+ T cell characterized in that it expresses a CXCR4^{Whim} mutation or a CXCR4 with the deletion in the C-terminal domain of between 10 and 20 amino acid residues and its use in therapy.

Persisting (central memory and effector memory), non-persisting (effector or exhausted subpopulations), anergic/tolerant, senescent and regulatory CD8+ T cells can be discriminated on their differential expression of surface markers including (but not limited to) CCR7, CD44, CD62L, CD122; CD127; IL15R, KLRG1, CD57, CD137, CD45RO, CD95, PD-1 CTLA, Lag3 and transcription factors such as T-bet/Eomes, BCL6, Blimp-1, STAT3/4/5 ID2/3, NFAT, FoxP3.

CD8+ T cells according to the present invention are primate CD8+ T cells, most preferably human CD8+ T cells.

The term “CXCR4”, for “C-X-C chemokine receptor type 4” refers to Ga protein-coupled receptor in the CXC chemokine receptor family specific for stromal-derived-factor-1 (SDF-1 also called CXCL12), a molecule endowed with potent chemotactic activity for lymphocytes. The sequence of said protein can be found under the Uniprot accession number P61073. CXCR-4 also known as fusin or CD184 (cluster of differentiation 184) is a protein of 352 amino acid residues that in humans is encoded by the cxcr4 gene (Gene ID: 7852).

The term “CXCR4^{Whim} mutation” refers to the autosomal dominant mutation associated with the rare combined primary immunodeficiency Warts, Hypogammaglobulinemia, Infections and Myelokathexis (WHIM) Syndrome (WS) which have been linked to inherited autosomal dominant gain-of-function mutations in CXCR4^{2,24}. These mutations result in the distal truncation of the C-term of CXCR4 and a desensitization- and internalization-resistant receptor in response to CXCL12^{3,6}. Patients also exhibit a severe, chronic pan-leukopenia with neutrophils, naive T cells and mature recirculating B cells being most affected⁷.

The different autosomal dominant gain-of-function mutations in CXCR4 associated with Whim syndrome described at this day include:
R334X (gene mutation 1000C>T) the most frequent mutation resulting in carboxy-terminal truncation of 19 residues,³
G336X, (gene mutation 1000C>T) resulting in carboxy-terminus truncation of 17 residues⁷,
E343X (gene mutation 1027G>T) resulting in carboxy-terminus truncation of the receptor of 10 residues,³
S339fs342X, (gene mutation 1016-1017delCT) resulting in carboxy-terminus truncation of the receptor of 13 residues and addition of 3 additional amino acid residues,^{3,4}
S338X (gene mutation 1013C>G), resulting in carboxy-terminus truncation of the receptor of 12 residues⁵
E343K (nucleic acid mutation 1027G>A)⁴

Accordingly in particular embodiment the CXCR4^{Whim} mutation is selected from the group consisting of: R334X, G336X, E343X, S341fs, S339fs342X, S338X, E343K.

The different autosomal dominant gain-of-function mutations in CXCR4 associated with Whim syndrome are located in the C-terminal domain of the CXCR4 receptor (see table 1), a domain responsible for regulation of the receptor (internalisation/inactivation).

Accordingly the C-terminal domain of the CXCR4 receptor, which is deleted in several Whim mutation could also be directly deleted to obtain the same biological effect (gain-of-function of CXCR4 associated with the long term CD8 memory response).

Biological activity of CD8 cells according to the invention (gain-of-function of CXCR4) can be measured for example with chemokine receptor internalization assay or cell migration after adding the CXCR4 agonist (SDF1/CXCL12) (as described in Balabanian K. et al Blood (2005) or in 5.

In a particular embodiment, a CXCR4 with the deletion in the C-terminal domain correspond to the deletion of the 10, 11, 12, 13, 14, 15, 16, 17, 18, 16, 17, 18, 19 20 amino acid residues in the C-terminal domain of the CXCR4 receptor.

TABLE 1
(from Liu et al Blood 2012⁴): Alignment of
predicted amino acid sequences from the
C-terminal domain of CXCR4WT and reported
of some of CXCR4 mutants associated
with WHIM syndrome.

| | |
|---|---|
| WT CXCR4 | 310 320 330 340 350 |
| | \| \| \| \| \| |
| | --KFKTSAQHALTSVSRGSSLKILSKGKRGGHSSVSTESESSSFHSS |
| E343K | --KFKTSAQHALTSVSRGSSLKILSKGKRGGHSSVSTKSESSSFESS |
| E343X | --KFKTSAQHALTSVSRGSSLKILSKGKRGGHSSVST |
| S341fs | --KPKTSAQHALTSVSRGSSLKILSKGKRGGHSSVPLSLSLQVFTPA . . . * |
| S339fs | --KFKTSAQHALTSVSRGSSLKILSKGKRGGHSCFH |
| S338X | --KFKTSAQHALTSVSRGSSLKILSKGKRGGH |
| G336X | --KFKTSAQHALTSVSRGSSLKILSKGKRG |
| R334X | --KFKTSAQHALTSVSRGSSLKILSKGK |

* Missense mutation that extends the sequence by an additional 10 amino acids, not shown for clarity

In a preferred embodiment the CXCR4^{Whim} mutation is R334X and S338X

The term “gene” refers to a natural or synthetic polynucleotide containing at least one open reading frame that is capable of encoding a particular polypeptide or protein after being transcribed or translated.

In one embodiment, the gene encoding CXCR4 with a neo cassette have been partially deleted or mutated (with a WHIM mutation) in order to partially delete the C-terminal domain of the CXCR4 receptor (as described in Balabanian, Blood 2005⁵). Briefly, the CXCR4 mutation is introduced in the CXCR4 coding region by PCR and confirmed by sequence analysis. The mutated CXCR4 cDNAs is cloned into the pTRIP vector and is expressed following a lentiviral-based strategy in activated PBMCs from healthy individuals.

As used herein, the term “mutated gene” means a gene in which a mutation has occurred. The term “mutation” as used herein means a change in the sequence of a nucleic acid and includes a base substitution, insertion, deletion, inversion, duplication, translocation, and the like used in genetics. The region of the mutation in a mutated gene is not limited to a transcriptional region, but includes a regulatory region such as a promoter which is required for gene expression.

Another object of the present invention relates to a population of T cells of the invention for use in therapy.

In some embodiment, the T cell is CD8+ T cell, CD4+ T cell or Gamma delta (γδ) T cell.

In some embodiment, the T cell is CD8+ T cell.

Thus, the present invention relates to a population of CD8 T cells of the invention for use in therapy.

As used herein, the term “population” refers to a population of cells, wherein the majority (e.g., at least about 50%, preferably at least about 60%, more preferably at least about 70%, and even more preferably at least about 80%) of the total number of cells have the specified characteristics of the cells of interest and express the markers of interest.

An ex vivo method for obtaining said population of T cells of the invention (with Whim mutation), may comprise the following step:
i) Obtaining a biological sample from a subject;
ii) Isolating T cells from said sample;
iii) In vitro expansion of T cells
iv) Modifying genetically said isolated T cells in order to silence or inactivate CXCR4 receptor in its C-terminal part.

In some embodiment, the T cell is CD8+ T cell, CD4+ T cell or Gamma delta (γδ) T cell.

As used herein, the term “subject” denotes a mammal, such as a rodent, a feline, a canine, and a primate. Preferably, a subject according to the invention is a human.

As used herein, the term “biological sample” refers to any body fluid or tissue. In one embodiment, the biological sample is blood sample.

As used herein, “isolating” refers to removal of a cell or a cell population from its natural environment. As used herein, “isolated” refers to a cell or a cell population that is removed from its natural environment (such as the blood sample) and that is isolated, purified or separated, and is at least about 75% free, 80% free, 85% free and preferably about 90%, 95%, 96%, 97%, 98%, 99% free, from other cells with which it is naturally present.

As used herein, the term “modifying genetically” refers to the addition, suppression or substitution of at least one nucleic acid in the genetic material of the cell.

According to the method of the present invention, the T cells of the invention are isolated from the sample. All the techniques known by the skilled man may be used.

In some embodiment, the T cells is CD8+ T cells.

In one embodiment, the CD8 T cells are isolated by cell sorter after pre-enrichment of CD8⁺ T cells by depletion of CD4⁺ and CD19⁺ cells. The purity of sorted CD8 cells is >97%.

According to the present invention, the T cell of the invention is genetically modified in order to delete or mutate the C-terminal domain of the CXCR4 receptor. In particular, the gene coding for CXCR4 is partially deleted or mutated resulting on the truncation of receptor CXCR4 in its C-terminal part and a desensitization- and internalization-resistant receptor in response to CXCL12.

In some embodiment, the T cells is CD8+ T cells.

All the techniques known by the skilled man may be used for partially deleting or for mutating the CXCR4 gene.

The gene encoding CXCR4 partially deleted (at C terminal tail) or mutated (with a WHIM mutation) may be delivered in vivo alone or in association with a vector. In its broadest sense, a “vector” is any vehicle capable of facilitating the transfer of the nucleic acid to the cells and typically CD8 cells. Classically, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of gene encoding CXCR4 partially deleted or mutated (with a WHIM mutation). Viral vectors are a preferred type of vector and include, but are not limited to nucleic acid sequences from the following viruses: retrovirus, such as moloney murine leukemia virus, harvey murine sarcoma virus, murine mammary tumor virus, and rous sarcoma virus; adenovirus, adeno-associated virus; SV40-type viruses; polyoma viruses; Epstein-Barr viruses; papilloma viruses; herpes virus; vaccinia virus; polio virus; and RNA virus such as a lentivirus. One can readily employ other vectors not named but known to the art.

Preferred viral vectors are based on non-cytopathic eukaryotic viruses in which non-essential genes have been replaced with the gene of interest. Non-cytopathic viruses include retroviruses (e.g., lentivirus), the life cycle of which involves reverse transcription of genomic viral RNA into DNA with subsequent proviral integration into host cellular DNA. Retroviruses have been approved for human gene therapy trials. Most useful are those retroviruses that are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell line with plasmid, production of recombinant retroviruses by the packaging cell line, collection of viral particles from tissue culture media, and infection of the target cells with viral particles) are provided in Kriegler, 1990 and in Murry, 1991. Preferred viruses for certain applications are the adeno-viruses and adeno-associated viruses, which are double-stranded DNA viruses. The adeno-associated virus can be engineered to be replication deficient and is capable of infecting a wide range of cell types and species. It has further advantages such as, heat and lipid solvent stability; high transduction frequencies in cells of diverse lineages, including hematopoietic cells; and lack of superinfection inhibition thus allowing multiple series of transductions. Reportedly, the adeno-associated virus can integrate into human cellular DNA in a site-specific manner, thereby minimizing the possibility of insertional mutagenesis and variability of inserted gene expression characteristic of retroviral infection. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion. Other vectors include plasmid vectors. Plasmid vectors have been extensively described in the art and are well known to those of skill in the art. See e.g. Sambrook et al., 1989. In the last few years, plasmid vectors have been used as DNA vaccines for delivering antigen-encoding genes to cells in vivo. They are particularly advantageous for this because they do not have the same safety concerns as with many of the viral vectors. These plasmids, however, having a promoter compatible with the host cell, can express a peptide from a gene operatively encoded within the plasmid. Some commonly used plasmids include pBR322, pUC18, pUC19, pRC/CMV, SV40, and pBlueScript. Other plasmids are well known to those of ordinary skill in the art. Additionally, plasmids may be custom designed using restriction enzymes and ligation reactions to remove and add specific fragments of DNA. Plasmids may be delivered by a variety of parenteral, mucosal and topical routes. For example, the DNA plasmid can be injected by intramuscular, eye, intradermal, subcutaneous, or other routes. It may also be administered by intranasal sprays or drops, rectal suppository and orally. It may also be administered into the epidermis or a mucosal surface using a gene-gun. The plasmids may be given in an aqueous solution, dried onto gold particles or in association with another DNA delivery system including but not limited to liposomes, dendrimers, cochleate and microencapsulation.

In a preferred embodiment, the gene encoding CXCR4 partially deleted or mutated (with a WHIM mutation) is under the control of a heterologous regulatory region, e.g., a heterologous promoter or lymphocyte specific promoter.

In one embodiment, an endonuclease is used for introducing specific mutation in the cxcr4 gene. In one embodiment, the “CRISPR/Cas9” technology is used for introducing specific mutation in the cxcr4 gene.

As used herein, the term “CRISPR” has its general meaning in the art and refers to Clustered Regularly Interspaced Short Palindromic Repeats which are the segments of prokaryotic DNA containing short repetitions of base sequences. In bacteria the CRISPR/Cas loci encode RNA-guided adaptive immune systems against mobile genetic elements (viruses, transposable elements and conjugative plasmids). Three types (I-III) of CRISPR systems have been identified. CRISPR clusters contain spacers, the sequences complementary to antecedent mobile elements. CRISPR clusters are transcribed and processed into mature CRISPR (Clustered Regularly Interspaced Short Palindromic Repeats) RNA (crRNA). The CRISPR-associated endonuclease, Cas9, belongs to the type II CRISPR/Cas system and has strong endonuclease activity to cut target DNA. Cas9 is guided by a mature crRNA that contains about 20 base pairs (bp) of unique target sequence (called spacer) and a trans-activated small RNA (tracrRNA) that serves as a guide for ribonuclease Ill-aided processing of pre-crRNA. The crRNA:tracrRNA duplex directs Cas9 to target DNA via complementary base pairing between the spacer on the crRNA and the complementary sequence (called protospacer) on the target DNA. Cas9 recognizes a trinucleotide (NGG) protospacer adjacent motif (PAM) to specify the cut site (the 3rd nucleotide from PAM). The crRNA and tracrRNA can be expressed separately or engineered into an artificial fusion small guide RNA (sgRNA) via a synthetic stem loop to mimic the natural crRNA/tracrRNA duplex. Such sgRNA, like shRNA, can be synthesized or in vitro transcribed for direct RNA transfection or expressed from U6 or HI-promoted RNA expression vector, although cleavage efficiencies of the artificial sgRNA are lower than those for systems with the crRNA and tracrRNA expressed separately. In some embodiments, the CRISPR-associated endonuclease can be a Cas9 nuclease. The Cas9 nuclease can have a nucleotide sequence identical to the wild type Streptococcus pyrogenes sequence. In some embodiments, the CRISPR-associated endonuclease can be a sequence from other species, for example other Streptococcus species, such as thermophilus; Pseudomona aeruginosa, Escherichia coli, or other sequenced bacteria genomes and archaea, or other prokaryotic microogranisms. Alternatively, the wild type Streptococcus pyrogenes Cas9 sequence can be modified. The nucleic acid sequence can be codon optimized for efficient expression in mammalian cells, i.e., “humanized.” A humanized Cas9 nuclease sequence can be for example, the Cas9 nuclease sequence encoded by any of the expression vectors listed in Genbank accession numbers KM099231.1 GL669193757; KM099232.1 GL669193761; or KM099233.1 GL669193765. Alternatively, the Cas9 nuclease sequence can be for example, the sequence contained within a commercially available vector such as PX330 or PX260 from Addgene (Cambridge, MA). In some embodiments, the Cas9 endonuclease can have an amino acid sequence that is a variant or a fragment of any of the Cas9 endonuclease sequences of Genbank accession numbers KM099231.1 GL669193757; KM099232.1; GL669193761; or KM099233.1 GL669193765 or Cas9 amino acid sequence of PX330 or PX260 (Addgene, Cambridge, MA). The Cas9 nucleotide sequence can be modified to encode biologically active variants of Cas9, and these variants can have or can include, for example, an amino acid sequence that differs from a wild type Cas9 by virtue of containing one or more mutations (e.g., an addition, deletion, or substitution mutation or a combination of such mutations). One or more of the substitution mutations can be a substitution (e.g., a conservative amino acid substitution). For example, a biologically active variant of a Cas9 polypeptide can have an amino acid sequence with at least or about 50% sequence identity (e.g., at least or about 50%, 55%, 60%, 65%, 70%, 75%, 80%, 85%, 90%, 95%, 97%, 98%, or 99% sequence identity) to a wild type Cas9 polypeptide. Conservative amino acid substitutions typically include substitutions within the following groups: glycine and alanine; valine, isoleucine, and leucine; aspartic acid and glutamic acid; asparagine, glutamine, serine and threonine; lysine, histidine and arginine; and phenylalanine and tyrosine. The Cas9 nuclease sequence can be a mutated sequence. For example the Cas9 nuclease can be mutated in the conserved FiNH and RuvC domains, which are involved in strand specific cleavage. For example, an aspartate-to-alanine (D10A) mutation in the RuvC catalytic domain allows the Cas9 nickase mutant (Cas9n) to nick rather than cleave DNA to yield single-stranded breaks, and the subsequent preferential repair through HDR can potentially decrease the frequency of unwanted indel mutations from off-target double-stranded breaks. The polypeptides that are biologically active variants of a CRISPR-associated endonuclease can be characterized in terms of the extent to which their sequence is similar to or identical to the corresponding wild-type polypeptide. For example, the sequence of a biologically active variant can be at least or about 80% identical to corresponding residues in the wild-type polypeptide. For example, a biologically active variant of a CRISPR-associated endonuclease can have an amino acid sequence with at least or about 80% sequence identity (e.g., at least or about 85%, 90%, 95%, 97%, 98%, or 99% sequence identity) to a CRISPR-associated endonuclease or to a homolog or ortholog thereof. A biologically active variant of a CRISPR-associated endonuclease polypeptide will retain sufficient biological activity to be useful in the present methods. The biologically active variants will retain sufficient activity to function in targeted DNA cleavage. The biological activity can be assessed in ways known to one of ordinary skill in the art and includes, without limitation, in vitro cleavage assays or functional assays.

It has already been successfully used to target important genes in many cell lines and organisms, including human (Mali et al., 2013, Science, Vol. 339: 823-826), bacteria (Fabre et al., 2014, PLoS Negl. Trop. Dis., Vol. 8:e2671.), zebrafish (Hwang et al., 2013, PLoS One, Vol. 8:e68708.), C. elegans (Hai et al., 2014 Cell Res. doi: 10.1038/cr.2014.11.), bacteria (Fabre et al., 2014, PLoS Negl. Trop. Dis., Vol. 8:e2671.), plants (Mali et al., 2013, Science, Vol. 339: 823-826), Xenopus tropicalis (Guo et al., 2014, Development, Vol. 141: 707-714.), yeast (DiCarlo et al., 2013, Nucleic Acids Res., Vol. 41: 4336-4343.), Drosophila (Gratz et al., 2014 Genetics, doi:10.1534/genetics.113.160713), monkeys (Niu et al., 2014, Cell, Vol. 156: 836-843.), rabbits (Yang et al., 2014, J. Mol. Cell Biol., Vol. 6: 97-99.), pigs (Hai et al., 2014, Cell Res. doi: 10.1038/cr.2014.11.), rats (Ma et al., 2014, Cell Res., Vol. 24: 122-125.) and mice (Mashiko et al., 2014, Dev. Growth Differ. Vol. 56: 122-129.).

In some embodiment, the endonuclease is CRISPR-Cpf1 which is the more recently characterized CRISPR from Provotella and Francisella 1 (Cpf1) in Zetsche et al. (“Cpf1 is a Single RNA-guided Endonuclease of a Class 2 CRISPR-Cas System (2015); Cell; 163, 1-13).

The endonuclease CRISPR/Cas9 may be delivered in vivo alone or in association using a viral-derived vector systems as described in WO2017068077.

T Cells of the Invention Expressing Chimeric Antigen Receptor

A further object of the present invention relates to the T cell of the invention characterized in that it expresses a chimeric antigen receptor which recognizes/binds to an antigen.

In some embodiment, the T cell is CD8+ T cell, CD4+ T cell or Gamma delta (γδ) T cell. In some embodiment, the T cell is CD8+ T cells.

Thus, the present invention relates to the CD8+ T cell of the invention characterized in that it expresses a chimeric antigen receptor which recognizes/binds to an antigen.

The present invention also relates to the T cell of the invention characterized in that it expresses a chimeric antigen receptor which recognizes/binds to an antigen for use in therapy.

The term “Chimeric Antigen Receptor” or “CAR” has its general meaning in the art and refers to an artificially constructed hybrid protein or polypeptide containing the antigen binding domains of an antibody (e.g., scFv) linked to T-cell signaling domains. In the context of the invention, the antigen binding domains of the antibody recognizes/binds to an antigen.

As used herein, the term “recognizes” or “binds” means in the context of the invention that the chimeric antigen receptor has affinity for an antigen.

The term “antigen” (“Ag”) as used herein refers to protein, peptide, nucleic acid (e.g. DNA plasmid) or tissue or cell preparations capable of eliciting a T cell response. In some embodiments, said antigen is a protein which can be obtained by recombinant DNA technology or by purification from different tissue or cell sources. Such proteins are not limited to natural ones, but also include modified proteins or chimeric constructs, obtained for example by changing selected amino acid sequences or by fusing portions of different proteins. The skilled person in the art will be able to select the appropriate antigen, depending on the desired T cell stimulation.

In some embodiments, the antigen is a protein or peptide coded by a DNA or other suitable nucleic acid sequence which has been introduced in cells by transfection, lentiviral or retroviral transduction, mini-gene transfer or other suitable procedures. In some embodiments, said antigen is a protein which can be obtained by recombinant DNA technology or by purification from different tissue or cell sources. Typically, said protein has a length higher than 10 amino acids, preferably higher than 15 amino acids, even more preferably higher than 20 amino acids with no theoretical upper limit. Such proteins are not limited to natural ones, but also include modified proteins or chimeric constructs, obtained for example by changing selected amino acid sequences or by fusing portions of different proteins. In some embodiments, said antigen is a synthetic peptide. Typically, said synthetic peptide is 3-40 amino acid-long, preferably 5-30 amino acid-long, even more preferably 8-20 amino acid-long. Synthetic peptides can be obtained by Fmoc biochemical procedures, large-scale multipin peptide synthesis, recombinant DNA technology or other suitable procedures. Such peptides are not limited to natural ones, but also include modified peptides, post-translationally modified peptides or chimeric peptides, obtained for example by changing or modifying selected amino acid sequences or by fusing portions of different proteins.

In a particular embodiment, the antigen is a tumoral antigen or a tumor-associated antigen (TAA).

As used herein, the term “tumoral antigen” refers to a tumoral antigen, or an active fragment thereof, that is recognized by the immune system.

Tumoral-antigens comprise, but are not limited to, cellular proteins, phosphoproteins, cellular surface proteins, cellular lipids, nucleic acids, glycoproteins, including cell surface receptors.

Examples of TAAs include, without limitation, melanoma-associated Ags (Melan-A/MART-1, MAGE-1, MAGE-3, TRP-2, melanosomal membrane glycoprotein gp100, gp75 and MUC-1 (mucin-1) associated with melanoma); CEA (carcinoembryonic antigen) which can be associated, e.g., with ovarian, melanoma or colon cancers; folate receptor alpha expressed by ovarian carcinoma; free human chorionic gonadotropin beta (hCGP) subunit expressed by many different tumors, including but not limited to ovarian tumors, testicular tumors and myeloma; HER-2/neu associated with breast cancer; encephalomyelitis antigen HuD associated with small-cell lung cancer; tyrosine hydroxylase associated with neuroblastoma; prostate-specific antigen (PSA) associated with prostate cancer; CA125 associated with ovarian cancer; and the idiotypic determinants of a B-cell lymphoma that can generate tumor-specific immunity (attributed to idiotype-specific humoral immune response), Mesothelin associated with pancreatic, ovarian and lung cancer, P53 associated with ovarian, colorectal, non small cell lung cancer, NY-ESO-1 associated with testis, ovarian cancer, EphA2 associated with breast, prostate, lung cancer, EphA3 associated with colorectal carcinoma, Survivin associated with lung, breast, pancreatic, ovarian cancer, HPV E6 and E7 associated with cervical cancer, EGFR associated with NSCL cancer. Moreover, Ags of human T cell leukemia virus type 1 have been shown to induce specific cytotoxic T cell responses and anti-tumor immunity against the virus-induced human adult T-cell leukemia (ATL). Other leukemia Ags can equally be used.

Tumor-associated antigens which can be used in the present invention are disclosed in the book “Categories of Tumor Antigens” (Hassane M. et al Holland-Frei Cancer Medicine (2003). 6th edition.) and the review Gregory T. et al (“Novel cancer antigens for personalized immunotherapies: latest evidence and clinical potential” Ther Adv Med Oncol. 2016; 8(1): 4-31) all of which are herein incorporated by reference.

Another object relates to a population of T cells of the invention characterized in that it expresses a chimeric antigen receptor which recognizes/binds to an antigen.

In some embodiment, the T cell is CD8+ T cells.

Thus, the present invention relates to a population of CD8+ T cell of the invention characterized in that it expresses a chimeric antigen receptor which recognizes/binds to an antigen.

Another object of the present invention relates to a method of producing the T cell of the invention expressing a chimeric antigen receptor which recognizes/binds to an antigen, which comprises the step of transfecting or transducing a T cell of the invention in vitro or ex vivo with a vector encoding for the chimeric antigen receptor.

In some embodiment, the T cell is CD8+ T cells.

Thus, the present invention relates to a method of producing the CD8+ T cell of the invention expressing a chimeric antigen receptor which recognizes/binds to an antigen, which comprises the step of transfecting or transducing a CD8+ T cell of the invention in vitro or ex vivo with a vector encoding for the chimeric antigen receptor.

The term “transduction” or “transducing” refers to the viral transfer of genetic material and its expression in a recipient cell.

The term “transfection” or “transfecting” as used herein refers to the process of introducing DNA (e.g., formulated DNA expression vector) into a cell, thereby, allowing cellular transformation.

As used herein, the term “vector” refers to a nucleic acid molecule allowing insertion of foreign nucleic acid without disrupting the ability of the vector to replicate and/or integrate in a host cell.

Methods of Treatment

The T cells populations of the present invention (population of T cells characterized in that it does express CXCR4 mutated according to the invention and population of T cells characterized in that it does express CXCR4 mutated according to the invention and in that it expresses a chimeric antigen receptor which recognizes/binds to an antigen), and more particular the CD8+ T cells population of the invention, are particularly suitable for therapeutic uses.

Accordingly, a further object of the present invention relates to the population of T cells characterized in that it does expresses CXCR4 mutated according to the invention and/or the population of T cells characterized in that it does expresses CXCR4 mutated according to the invention and in that it expresses a chimeric antigen receptor which recognizes/binds to an autoantigen for use in adoptive cell therapy in a subject in need thereof. In some embodiment, the T cell is CD8+ T cells.

Thus, the present invention relates to the population of CD8+ T cells characterized in that it does expresses CXCR4 mutated according to the invention and/or the population of CD8+ T cells characterized in that it does expresses CXCR4 mutated according to the invention and in that it expresses a chimeric antigen receptor which recognizes/binds to an autoantigen for use in adoptive cell therapy in a subject in need thereof.

The term “adoptive cell therapy” as used herein refers to a cell-based immunotherapy that relates to the transfusion of autologous or allogenic lymphocytes, genetically modified or not. For the purpose of the present invention, the CD8 T cells are genetically modified.

The populations of T cells of the present invention, and more particularly the population of CD8+ T cells of the present invention, can be utilized in methods and compositions for adoptive cell therapy in accordance with known techniques, or variations thereof that will be apparent to those skilled in the art based on the instant disclosure. See, e.g., US Patent Application Publication No. 2003/0170238 to Gruenberg et al; see also U.S. Pat. No. 4,690,915 to Rosenberg. In some embodiments, the cells are formulated by first harvesting them from their culture medium, and then washing and concentrating the cells in a medium and container system suitable for administration (a “pharmaceutically acceptable” carrier) in a treatment-effective amount. Suitable infusion medium can be any isotonic medium formulation, typically normal saline, Normosol R (Abbott) or Plasma-Lyte A (Baxter), but also 5% dextrose in water or Ringer's lactate can be utilized. The infusion medium can be supplemented with human serum albumin. A treatment-effective amount of cells in the composition is dependent on the age and weight of the recipient, on the severity of the targeted condition. The number of cells will depend upon the ultimate use for which the composition is intended, as will the type of cells included therein. The clinically relevant number of immune cells can be apportioned into multiple infusions that cumulatively equal or exceed the desired total amount of cells.

For the purpose of the invention, the T cells used in the adoptive cell therapy may be isolated from the subject (“autologous cells”) or from another individual (“allogeneic cells”).

In some embodiment, the T cells is CD8+ T cells.

As used herein, “allogeneic cells” refers to cells isolated from one subject (the donor) and infused in another (the recipient or host).

As used herein, “autologous cells” refers to cells that are isolated and infused back into the same subject (recipient or host).

In one embodiment, the T cells used in the adoptive cell therapy may derived from stem cells.

In some embodiment, the T cells is CD8+ T cells.

The terms “stem cell” as used herein, refer to a cell in an undifferentiated or partially differentiated state that has the property of self-renewal and has the developmental potential to differentiate into multiple cell types, without a specific implied meaning regarding developmental potential (i.e., totipotent, pluripotent, multipotent, etc.).

In a particular embodiment, the T cells used in the adoptive cell therapy may derived from induced pluripotent stem cells.

In some embodiment, the T cells is CD8+ T cells.

As used herein, the terms “iPSC” and “induced pluripotent stem cell” are used interchangeably and refers to a pluripotent stem cell artificially derived (e.g., induced or by complete reversal) from a non-pluripotent cell, typically an adult somatic cell, for example, by inducing a forced expression of one or more genes.

In a particular embodiment, the T cells used in the adoptive cell therapy may derived from embryonic stem cells.

In some embodiment, the T cells is CD8+ T cells.

The term “embryonic stem cell” as used herein refers to naturally occurring pluripotent stem cells of the inner cell mass of the embryonic blastocyst (see, for e.g., U.S. Pat. Nos. 5,843,780; 6,200,806; 7,029,913; 7,584,479, which are incorporated herein by reference). Such cells can similarly be obtained from the inner cell mass of blastocysts derived from somatic cell nuclear transfer (see, for example, U.S. Pat. Nos. 5,945,577, 5,994,619, 6,235,970, which are incorporated herein by reference). Embryonic stem cells are pluripotent and give rise during development to all derivatives of the three primary germ layers: ectoderm, endoderm and mesoderm. In other words, they can develop into each of the more than 200 cell types of the adult body when given sufficient and necessary stimulation for a specific cell type. They do not contribute to the extra-embryonic membranes or the placenta, i.e., are not totipotent.

In one embodiment, the CD8 T cells used in the adoptive cell therapy may derived from the conversion of conventional CD4+ T cells.

A further object of the present invention relates to a method of treating infectious disease or a cancer in a subject in need thereof, said method comprising administering to the subject a therapeutically effective amount of the population of T cells characterized in that it does expresses CXCR4 mutated according to the invention and/or the population of T cells characterized in that it does expresses CXCR4 mutated according to the invention and in that it expresses a chimeric antigen receptor which recognizes/binds to an antigen.

In some embodiment, the population of T cell is a population of CD8+ T cells.

As used herein, the term “infectious disease” refers to a condition in which an infectious organism or agent is present in a detectable amount in the blood or in a normally sterile tissue or normally sterile compartment of a subject. Infectious organisms and agents include viruses, mycobacteria, bacteria, fungi, and parasites. The terms encompass both acute and chronic infections, as well as sepsis. In a particular embodiment the infectious organism is a virus or a bacteria that is responsible of lung infection such as:

Viral lung infections: Adenovirus, Influenza A virus, Influenza B virus, Human parainfluenza viruses, Human respiratory syncytial virus, SARS coronavirus, Middle East respiratory syndrome coronavirus.

Bacterial lung Infections: Haemophilus influenza, Staphylococcus aureus, Klebsiella pneumoniae, Legionella pneumophila, Mycoplasma pneumoniae, Chlamydophila pneumoniae, Chlamydia psittaci, . . . .

As used herein, the term “cancer” refers to a condition in which abnormally replicating cells of host origin are present in a detectable amount in a subject. The cancer can be a malignant or non-malignant cancer. Cancers include, but are not limited to, biliary tract cancer; brain cancer; breast cancer; cervical cancer; choriocarcinoma; colon cancer; endometrial cancer; esophageal cancer; gastric cancer; intraepithelial neoplasms; leukemias; lymphomas; liver cancer; lung cancer; melanoma; neuroblastomas; oral cancer; ovarian cancer; pancreatic cancer; prostate cancer; rectal cancer; renal cancer; sarcomas; skin cancer; testicular cancer; thyroid cancer; as well as other carcinomas and sarcomas. Cancers can be primary or metastatic.

In a particular embodiment the cancer according to the invention is due to an unregulated growth of hematopoietic cells or undifferentiated hematopoietic bone marrow cells (hematopoietic stem cell).

As intended herein the expression “hematopoietic stem cell (HSC)” refers to adult multipotent stem cells that give rise to all the blood cell types including for example myeloid lineages (monocytes and macrophages, neutrophils, basophils, eosinophils), erythrocytes, megakaryocytes/platelets, and lymphoid lineages (T-cells, B-cells, NK-cells).

The expression “hematopoietic stem cell malignancy” or “hematopoietic malignancy” according to the invention includes, but are not limited to, acute myeloid leukemia (AML), acute lymphoblastic leukemia, Chronic myeloid, lymphoid leukemia, lymphoma myelodysplastic syndrome and Adult T cell leukemia (as defined in 2008 WHO classification).

As used herein, the term “subject” denotes a mammal, such as a rodent, a feline, a canine, and a primate. Preferably, a subject according to the invention is a human.

As used herein, “treatment” or “treating” is an approach for obtaining beneficial or desired results including clinical results. For purposes of this invention, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread of the disease, preventing or delaying the recurrence of the disease, delaying or slowing the progression of the disease, ameliorating the disease state, providing a remission (partial or total) of the disease, decreasing the dose of one or more other medications required to treat the disease, delaying the progression of the disease, increasing the quality of life, and/or prolonging survival. The term “treatment” encompasses the prophylactic treatment. As used herein, the term “prevent” refers to the reduction in the risk of acquiring or developing a given condition.

As used herein the terms “administering” or “administration” refer to the act of injecting or otherwise physically delivering a substance as it exists outside the body into the subject, such as by mucosal, intradermal, intravenous, subcutaneous, intramuscular delivery and/or any other method of physical delivery described herein or known in the art. When a disease, or a symptom thereof, is being treated, administration of the substance typically occurs after the onset of the disease or symptoms thereof. When a disease or symptoms thereof, are being prevented, administration of the substance typically occurs before the onset of the disease or symptoms thereof.

The “therapeutically effective amount” is determined using procedures routinely employed by those of skill in the art such that an “improved therapeutic outcome” results. It will be understood, however, that the total daily usage of the compositions of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination; and like factors well known in the medical arts.

According to the invention, the populations of T cells are administered to the subject in the form of a pharmaceutical composition.

Accordingly, a further object of the present invention relates to a pharmaceutical composition comprising the population of T cells characterized in that it expresses CXCR4 mutated according to the invention and/or the population of T cells characterized in that it expresses CXCR4 mutated according to the invention and in that it expresses a chimeric antigen receptor which recognizes/binds to an antigen.

In some embodiment, the population of T cell is a population of CD8+ T cells.

Typically, the populations of T cells, and in particular the population of CD8+ T cells, may be combined with pharmaceutically acceptable excipients, and optionally sustained-release matrices, such as biodegradable polymers, to form therapeutic compositions. “Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. In the pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms. In one embodiment, the CD8 T cells populations of the invention are administered by parenteral route. In a preferred embodiment, the CD8 T cells populations of the invention are administered by intravenous route. Typically, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Solutions of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms. The populations of T cell, and in particular the population of CD8 T cells, can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. Sterile injectable solutions are prepared by incorporating the active compounds in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the typical methods of preparation are vacuum-drying and freeze-drying techniques which yield a powder of the active ingredient plus any additional desired ingredient from a previously sterile-filtered solution thereof. The preparation of more, or highly concentrated solutions for direct injection is also contemplated, where the use of DMSO as solvent is envisioned to result in extremely rapid penetration, delivering high concentrations of the active agents to a small tumor area. Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. Some variation in dosage will necessarily occur depending on the condition of the subject being treated. The person responsible for administration will, in any event, determine the appropriate dose for the individual subject.

The invention will be further illustrated by the following figures and examples. However, these examples and figures should not be interpreted in any way as limiting the scope of the present invention.

### FIGURES

FIG. 1: Schematic View of the Materiel and Method

Schematic view of the experimental system used in the study. Left: WT mice and Whim mice (CXCR4^{1013/+}) were infected intranasally by 2·10⁵ PFU of Vaccinia Virus (VV). Right: naïve (CD44^{low}) CD8 T-cells were FACS-sorted from F5-WT (expressing CD45.1. congenic marker) and FS-Whim (expressing CD45.2 congenic marker) mice and were mixed in equal numbers in PBS. The equivalent of 1·10⁵ F5-WT and 1·10⁵ FS-Whim were injected intravenously into C57Bl/6 WT recipient (expressing both CD45.1 and CD45.2 congenic marker). 1 to 2 days following cells transfer, mice were infected intranasally by 2·10⁵ PFU of Vaccinia Virus expressing NP68 peptide (VV-NP68, that is specific for F5-TCR)

(FIG. 2 Delayed recirculation pattern of CD8 T-cells in Whim mice following infection. (a, b) WT and Whim mice were infected intranasally by Vaccinia Virus and absolute Numbers of total CD8 T-cells (gated as CD8⁺ CD44^{hi}) (a) or antigen-specific CD8 T-cells (gated as CD8⁺ CD44^{hi} B8R⁺ except for day 0, gated on total CD8⁺ B8R⁺) (b) were followed in the blood of infected animals, at indicated time points. (c) Absolute numbers of Antigen-specific CD8 T-cells (gated as CD8⁺ CD44^{hi} B8R⁺) in lung draining LN. White and black dots show WT and CXCR4 Whim mice, respectively. Data represent a pool of 3 (a, b) or 2 (c) independent experiments.

FIG. 3: WT and Whim mice were infected intranasally by Vaccinia Virus. (a) Absolute Numbers of total CD8 T-cells (gated as CD8⁺ CD44^{hi}) or antigen-specific CD8 T-cells (gated as CD8⁺ CD44^{hi} B8R⁺) were calculated by adding up corresponding cells counted in the medLN, spleen, BM and lung, 6 weeks following infection. (b) Distribution of total CD8 T-cells (top panel) or Antigen specific CD8 T-cells (lower panel) was evaluated as the fraction of total memory cells that is located in indicated organs. (c) Splenocytes from WT and Whim infected animals were stimulated in vitro for 5 h with B8R cognate peptide. % of cells producing indicated cytokines was evaluated by flow-cytometry within CD8⁺ CD44^{hi} gated cells. White and black dots show WT and CXCR4 Whim mice, respectively. Data represent a pool of 4 (a), 3 (b), or 1 (c) independent experiments with 4 (c) to 19 animals (a-b).

FIG. 4: NP68 specific F5-WT (CD45.1+) and F5-Whim (CD45.2+) naïve CD8 T-cells were co-transferred into congenic mice (CD45.1+CD45.2+) and further subjected to intranasal infection by Vaccinia Virus expressing NP68. Mice were analysed two to 3 months post-infection. (a) Absolute Numbers of total F5 CD8 T-cells were calculated by adding up corresponding cells counted in the medLN, spleen, BM and lung. (b) Splenocytes from infected animals containing F5-WT and FS-Whim CD8 T-cells) were stimulated in vitro for 5 h with NP68. % of cells producing IFNg was evaluated by flow-cytometry within F5-WT (CD45.1+ CD45.2−) and FS-Whim (CD45.2+ CD45.1−) CD8⁺ CD44^{hi} gated cells. (c) Distribution of F5-WT (white dots) and FS-Whim (Black dots) CD8 T-cells was evaluated as the fraction of total F5-WT or FS-Whim memory cells located in indicated organs.

White and black dots show WT and CXCR4 Whim mice, respectively. Data represent a pool of 3 (a,c) or 2 (b) independent experiments with at least 8 animals in total.

FIG. 5: Mice co-transferred with F5-WT and F5-Whim naïve CD8 T-cells were infected by VV-NP68 as previously described.
(a) F5-WT and—Whim CD8 T-cells were followed in the blood of infected animals, at indicated time points. Datas are indicated as the ratio between the number of F5-WT and the number of FS-Whim cells. Dotted line represents a ratio=1, indicating similar numbers of F5-WT and F5-Whim.(b) Absolute numbers of F5-WT and FS-Whim in the blood of infected animals, at indicated time points.(c) Absolute Numbers of total F5 CD8 T-cells were calculated by adding up corresponding cells counted in the medLN, spleen, BM and lung, day 578 post infection. (d) Absolute Numbers of total F5 CD8 T-cells in in the Bone Marrow (top panel) and the spleen (lower panel) at indicated time post infection.

White and black dots show FS-WT and FS-Whim, respectively. Data are representative of 3 independent experiments with at least 5 mice per experiment.

### EXAMPLE

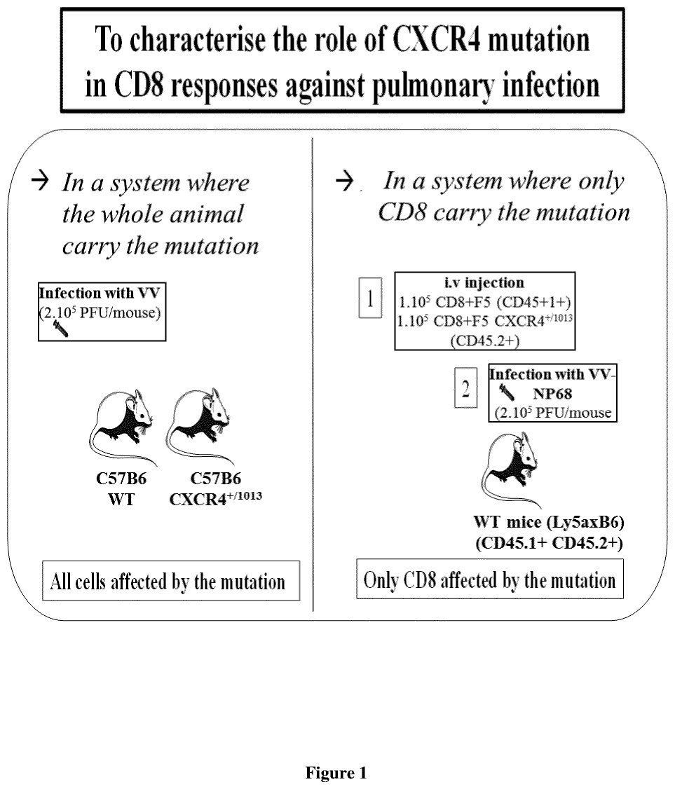

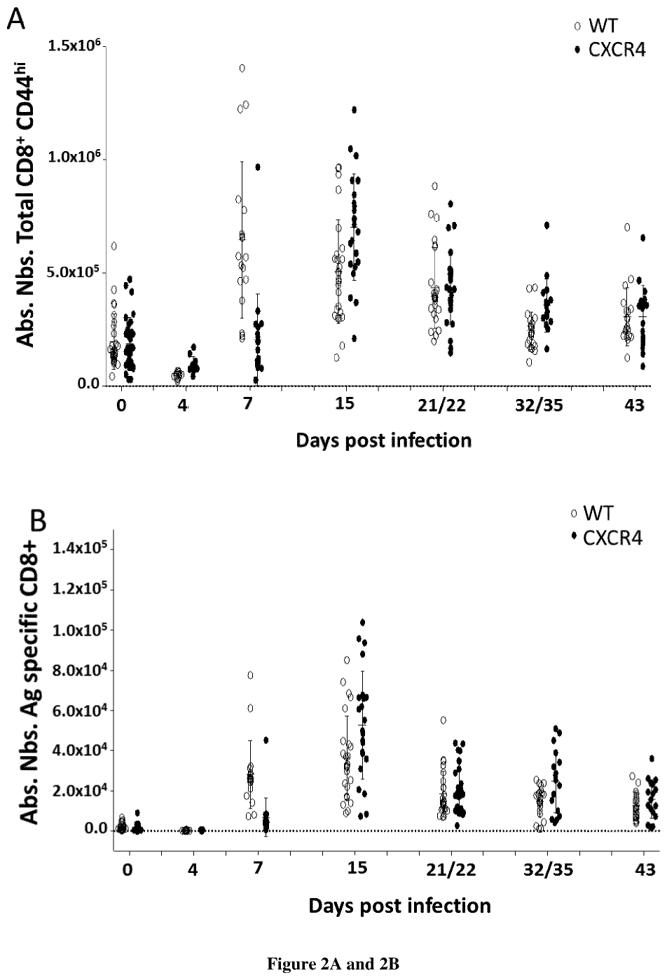

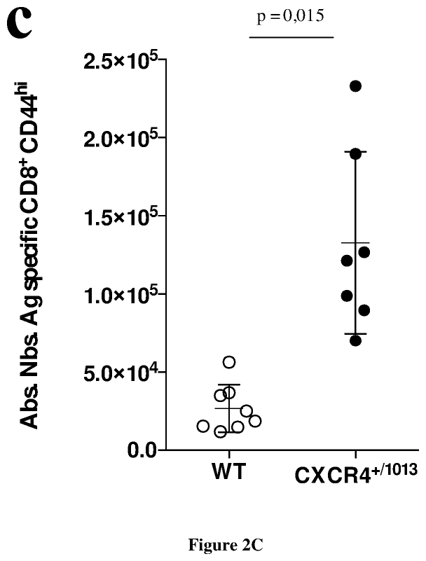

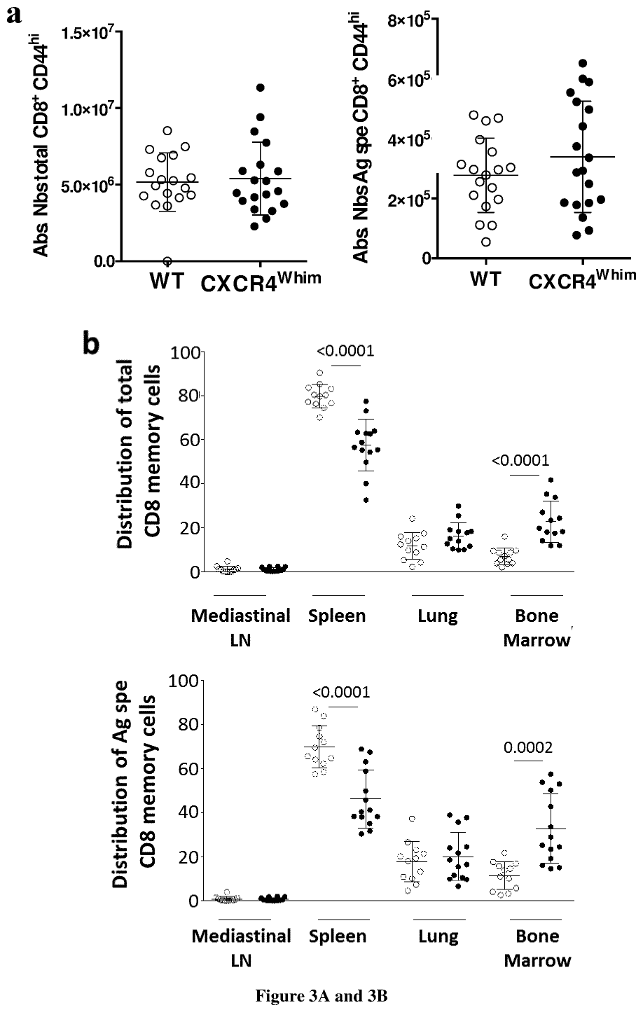

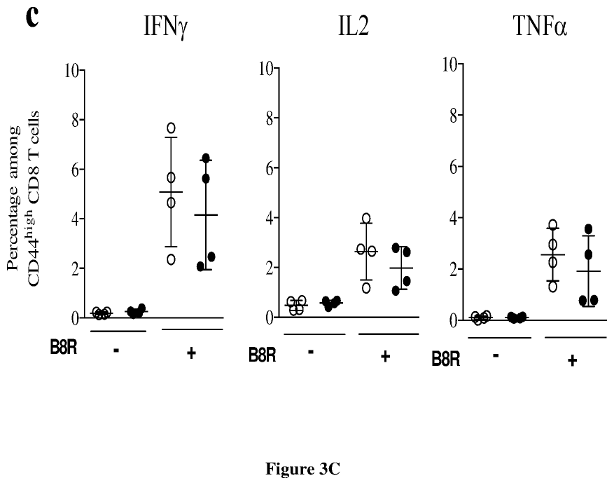

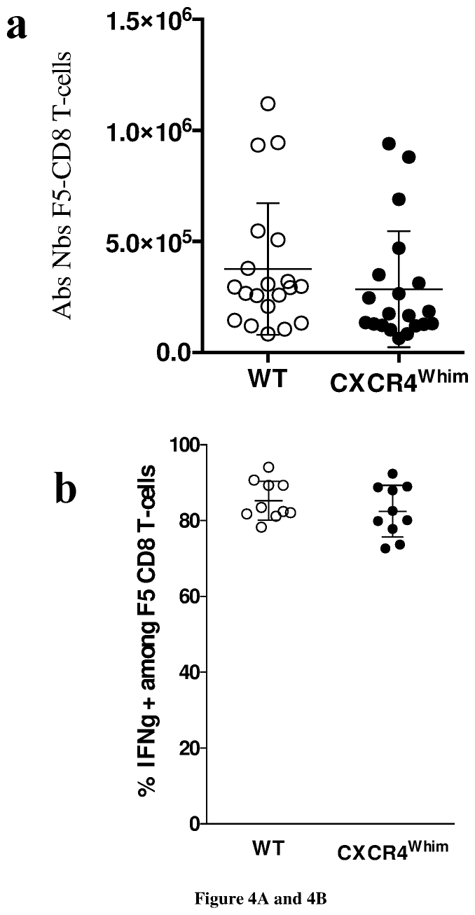

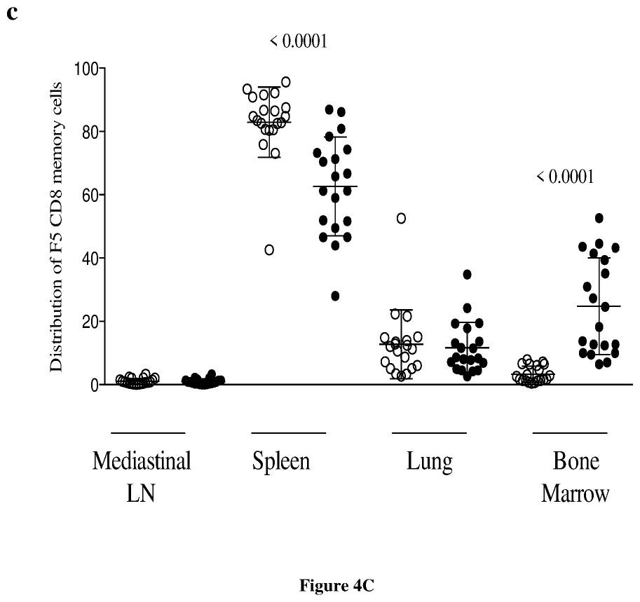

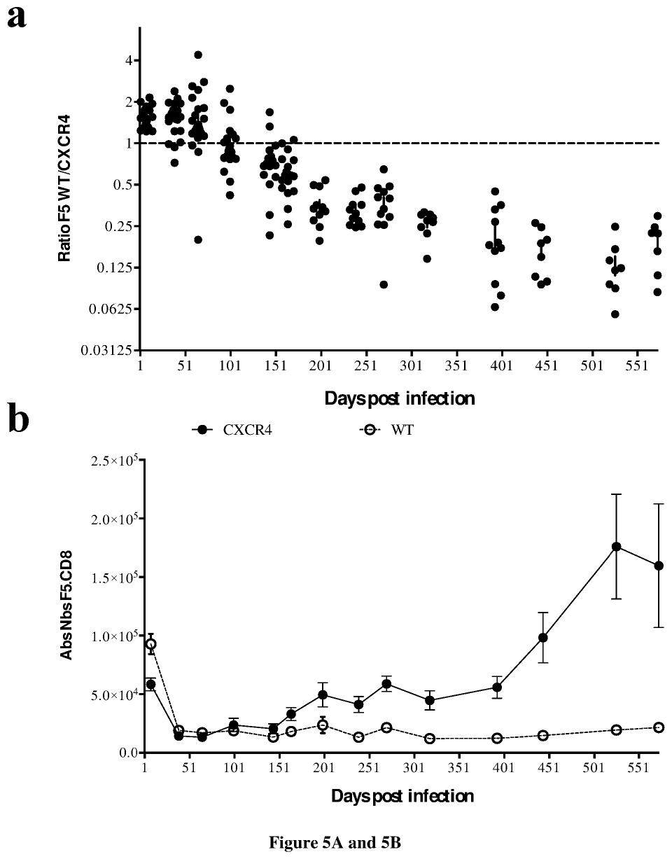

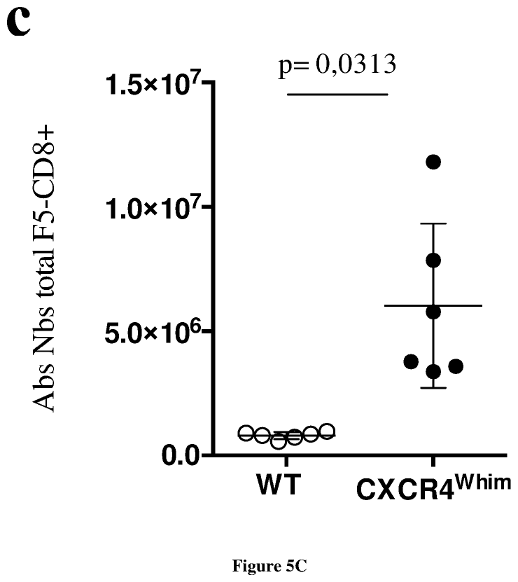

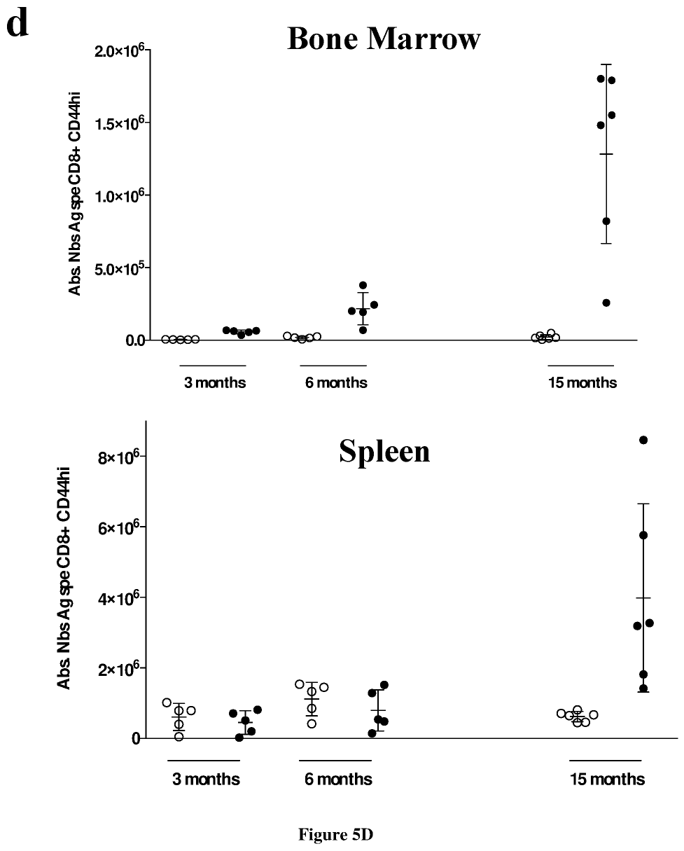

Material & Methods (see FIG. 1)

Mice:

C57BL/6 mice from Charles River were used as wild-type controls. C57BL/6 CXCR4^{+/1013} mice (Whim mice) were provided by Dr K. Balabanian⁶. F5 TCR-transgenic mice (CD45.1⁺) were obtained from Dr D. Kioussis²⁵. FS-CXCR4^{+/1013} (FS-Whim, CD45.2⁺) and C57BL/6.Ly5.1 (CD45.1⁺.CD45.2⁺) were generated in the animal house (PBES). All mice were bred in the PBES (SFR Biosciences animal facility, Lyon, France) under specific pathogen-free conditions. Experiments were done on mice aged from six weeks to 18 months (memory compartment). All animal procedures were approved by our local Animal Evaluation Committee (CECCAPP).

Virus and Immunization:

Recombinant Vaccinia Viruses (VV) expressing the NP68 epitope were engineered from VV (strain Western Reserve) by Dr Denise Yu-Lin Teoh from Pr Sir Andrew McMichael's laboratory at the Human Immunology unit, Institute of Molecular Medicine, Oxford, UK.

For cell transfer, C57BL/6 (CD45.1⁺.CD45.2⁺) recipients were transferred with 1·10⁵ CD8-Ts from F5-Whim and FS-WT donor mice, by intra-venous injection in the retro-orbital sinus. The following day, recipients were infected by intra-nasal injection with VV-NP68 (2·10⁵ PFU/mouse).

Cell Preparation, Culture and Activation:

Blood was collected from the retro-orbital sinus into 1 mL PBS containing 4 mM EDTA (Gibco). Flow-count fluorospheres (Beckman-Coulter) were then added to each tube and absolute numbers of cells/mL were calculated using the formula: [(Total number of cells/Total number of fluorospheres)×fluorosphere concentration]/volume.

Single cells suspension from mediastinal lymph node and spleen were obtained by mechanical disaggregation on a 100 μm cell strainer (BD Falcon). Lungs were flushed with PBS before harvesting from animals. To analyze the lung resident compartment, CD45 (clone 30-F11) was injected intra-venously before killing animals²⁶. Single cells suspensions were obtained using the lung dissociation kit, according to the manufacturer's instructions (Miltenyi Biotec). Cells from bone marrow were collected by flushing complete medium through tibias and femurs. For all organs, cells were resuspended in complete medium (DMEM, 6% Fetal Calf Serum, 1M hepes, 50 μg mL⁻¹ gentamicin, 50 μM β-mercapto-ethanol). Absolute numbers of lymphocytes were determined using Accuri C6 Flow instrument (BD Biosciences) and Cflow software.IFN-γ production by CD8-Ts was induced by restimulation with NP68 (10 nM) for five hours, in presence of brefeldin A (1/500 dilution).

Flow Cytometry:

Cells were stained for 30 minutes on ice, in PBS 0.5% BSA, 0.01% NaAzide. The following antibodies, coupled to the appropriate fluorochromes, were used: CD8 (clone 53-6.7), CD45.1 (A20), CD44 (IM7.8.1), from BD Biosciences; CD45 (30-F11), from Biolegend; CD45.2 (104), CD62L (MeI14), from eBiosciences.

To evaluate cytokine secretion, cells were permeabilized using Cytofix/cytoperm kit (BD Biosciences), before being incubated with an antibody against IFN-γ (XMG1.2, BD Biosciences).

All samples were acquired on LSR Fortessa flow cytometer (BD Biosciences) and analysed using FlowJo software (TreeStar).

Statistical Analysis:

Results were analysed with Prism software. Data are expressed as means+/−SEM. Statistical analyses used the unpaired two-tailed t-test and one-way or two-ways Anova. A p-value<0.05 was considered to be statistically significant.

Results

CD8 Responses to Lung Viral Infection are Slightly Delayed in Whim Mice

Upon infection, CD8 responses encompass substantial trafficking between SLO and infected tissue. Naïve CD8 T-cells are activated in the mediastinal LN (medLN, lungs draining LN) before re-entering blood circulation in order to migrate back to the lung. In order to analyse the role of CXCR4 on CD8 trafficking, we made use of a mouse model of Whim syndrome, carrying CXCR4+/1013 gain-of-function mutation (referred as Whim mice)⁶. Importantly and as described previously, this mouse model recapitulates immune defects observed in Whim patients, including decreased numbers of CD8 T-cells in the spleen and the blood of naïve animals, as compared to wild type (WT) animals, reflecting lymphopenia described in patients (⁶ and data not shown). In order to evaluate CD8 T-cells responses following lung viral infection, we performed intranasal infection of WT and Whim mice with Vaccinia Virus (VV). Blood analysis of WT infected animals indicate that absolute numbers of total activated CD8 T-cells (as characterized by the expression of CD44 activation marker) was increased in this compartment at day 7 post infection, probably reflecting their release from the draining LN (FIG. 2a). Interestingly, when similar analysis was made in the blood of Whim mice, we could not observe such an increase at day 7 post infection (FIG. 2a). However, comparable numbers of cells could be detected in the blood of both WT and Whim mice 15 days post-infection (although with a slight advantage for Whim animals) suggesting a delayed circulation pattern of activated CD8 T-cells, in Whim animals (FIG. 2a). In order to specifically assess the recirculation pattern of Antigen specific CD8 T-cells, we made use of tetramers that recognise B8R specific CD8 T-cells, since B8R is a dominant epitope of Vaccinia Virus in C57Bl/6 animals. FIG. 1b shows that the delayed recirculation pattern observed for total activated CD8 T-cells in Whim mice was also true for B8R specific CD8 T-cells, with similar kinetics. Since CXCL12, the major ligand for CXCR4, is largely secreted in the LNs, we hypothesised that CD8 T-cells could be sequestered in this compartment, in Whim animals. As anticipated, the delayed appearance of CD8 T-cells in the blood of Whim animals was associated with a corresponding retention of Antigen-specific cells in the mediastinal LN, at day 7 post infection (FIG. 2c). Altogether, those results suggest that CD8 responses can be generated in Whim mice, with a minor defect during the initial activation phase that delay the release of CD8 T-cells from the draining LN.

To further investigate the degree and nature of this delay, we performed mathematical modelling that estimated approximatively 1 to 2-day delay (data not shown).

CD8Whim Memory Cells are Preferentially Localised in the BM

We next thought to analyze the capacity of Whim mice to generate CD8 memory T-cells following lung infection. To that aim, we performed intranasal infection of WT and Whim animals with Vaccinia virus, and studied CD8 T-cells numbers and phenotype in the Lung, medLN and spleen, six weeks following infection. Since memory CD8 T-cells can home in the bone marrow (BM) and because this compartment is enriched for CXCL12, we included it in our analysis. By evaluating the sum of memory CD8 T-cells harvested from the spleen, the lung, the mediastinal LN and BM, we could show that comparable numbers of total and Ag-specific CD8 T-cells (specific for B8R) were generated in WT and Whim mice following infection (FIG. 3a, left and right panel, respectively). In addition, no differences could be observed with regards to their capacity to produce IFNγ, IL-2 and TNF-α (typical cytokines produced by memory CD8) when memory CD8 splenocytes were re-stimulated ex vivo with their cognate antigen (here B8R) (FIG. 3c). We next seek to understand whether CXCR4-Whim mutation could affect the localization of memory CD8 T-cells amongst the different organs. To that aim, we evaluate the fraction of total memory CD8 T-cells that could be harvested from the spleen, the lung, mediastinal LN and BM, in WT and Whim animals. Remarkably, distinct distribution was observed, with a highly preferential localization of CXCR4^{Whim} CD8 memory cells in the BM of infected animals, as compared to WT, and a respective decrease of cells in the spleen (FIG. 3b).

In order to assess whether this preferential localization of memory CD8 T-cells is the BM was CD8-cells intrinsic, we made use of F5 TCR transgenic mice whose TCR specifically recognise NP68 peptide²⁵. WT (FS-WT) expressing CD45.1 congenic marker and CXCR4+/1013 (FS-Whim, CD45.2+) naïve CD8 T-cells expressing F5 transgene were co-transferred in congenic host (CD45.1+CD45.2+) before intranasal infection with a recombinant Vaccinia Virus expressing NP-68 (VV-NP68)¹⁶. 60 days post infection, analysis of medLN, Lung, Spleen and BM compartment showed neither differences in absolute numbers between FS-WT and FS-Whim (FIG. 4a), nor in the capacity of spleen memory cells to respond to NP68 peptide ex vivo and produce IFNg (FIG. 4b).

Nevertheless and similar to Whim mice, FS-Whim memory cells showed a modified distribution as compared to FS-WT, with a substantial increase of cells in the BM (FIG. 4c). Of note this was also observed when FS-WT or FS-Whim were transferred in separate hosts. Altogether, those results suggest that CXCR4 Whim mutation within CD8 T-cells confers their preferential localisation in the BM, a site that is enriched for CXCL12.

CD8whim Memory Cells Outnumber WT Memory Cells in Lymphoid Organs Over Time

BM has been suggested as a preferential site for CD8 memory maintenance, but discrepancies exist regarding the effect of specific bone marrow niches on CD8 memory cells outcome.

Since we observed a preferential localization of CD8-whim in the BM, we next thought to investigate whether this could impact long-term maintenance and/or accumulation of CD8 memory cells. To that aim, we co-transferred FS-WT and FS-CXCR4 in naïve WT host and subsequently performed intranasal infection with VV-NP68 virus. Blood kinetics of FS-WT and FS-CXCR4 indicate comparable numbers of both cell types in the blood (ratio FS-WT/F5-CXCR4 close to 1), during early memory phase (FIG. 5A et 5B, until day 100). However, over time, blood was gradually populated by F5-CXCR4 and the ratio became in favour of F5-CXCR4 (FIG. 5A). Remarkably, analyses of cell numbers in the blood over time indicate that F5-WT numbers were maintained throughout time, while F5-CXCR4 numbers increased when the ratio started to be reverted (FIG. 5C). Even more interestingly, analyses of cell numbers in Spleen, Lungs and LN at very late time points (from day 300 post infection) showed a dramatic increase in F5-CXCR4 absolute numbers in all organs, while F5-WT CD8 numbers remain similar to early memory stage (FIG. 5 C). More specifically, numbers of FS-Whim cells gradually increased in the BM between 3 and 15 months post infection (FIG. 5D, top pannel), while their number started increasing in the spleen only between 6 and 15 months post infection (FIG. 5D, lower panel)

Molecular and Cellular Mechanism Underlying the Increase Number of CD8^{Whim} Memory Cells

To apprehend the relative contribution of BM localization in CD8 proliferation and/or survival of memory CD8 T-cells, current analysis includes:
BrDU assay (Chaix:2014gp) and cell cycle analysis combined with mathematical modelling;
scRNA sequencing²⁷ comparing F5-WT and FS-Whim isolated from the bone marrow, >10 months post infection with VV-NP68

Role of CXCR4^{Whim} in the Long-Term Maintenance of Anti-Tumor Responses.

Innovating immunotherapy approaches are currently developed and include Chimeric Antigen Receptor (CAR) that are engineered and transferred into T-cells to reprogram their cytotoxicity toward an antigen expressed by a given tumour²⁸. However, and although CAR-T cells therapies have given promising results thus far on Multiple Myeloma and Acute Lymphoblastic Leukemia, some patients still relapse^{29,30}, emphasizing the need for a better maintenance of CAR-T cells. Interestingly, CD8 T-cells over-expressing CXCR4 have shown a better protection in mice models of lymphoma, as compared to WT³¹. Since much insight related to the feature of current immunotherapies targeting CD8 and memory CD8 T-cells has come from the study of virus infection of mice (e.g. the role of PD1 in regulating CD8 T cells post LCMV infection³²), we study the impact of CXCR4 Whim-mutation on the long-term maintenance and pool size of tumor-specific CD8 T-cells responses.

This analysis include:
investigating anti-tumoral responses of Whim mice (as compared to WT) and mice transferred with F5-WT and F5-CXCR4^{Whim CD}8 T-cells following immunization with a tumor cell line expressing NP68. This study include the analysis of the long term maintenance of tumor-specific memory CD8 T-cells.
proof of concept of the benefit of CAR-T cells expressing CXCR4^{Whim} for the long-term maintenance of CD8 responses against a tumor

TABLE 2
Useful amino acid sequences for
practicing the invention

| SEQ ID NO | Amino acid sequence |
|---|---|
| 1. Cterm | KFKTSAQHALTSVSRGSSLK |
| domain WT | ILSKGKRGGHSSVST |
| CKCR4 | ESESSSFHSS |
| 2. Cterm | KFKTSAQHALTSVSRGSSLK |
| domain of | ILSKGKRGGHSSVST |
| E343K | KSESSSFHSS |
| 3. Cterm | KFKTSAQHALTSVSRGSSL |
| domain of | KILSKGKRGGHSSVST |
| E343X | |
| 4. Cterm | KFKTSAQHALTSVSRGSS |
| domain of | LKILSKGKRGGHSSVPL |
| E341fs | SLSLQVFTPA . . . * |
| 5. Cterm | KFKTSAQHALTSVSRGSS |
| domain of | LKILSKGKRGGHSCFH |
| E339fs | |
| 6 Cterm | KFKTSAQHALTSVSRGS |
| domain of | SLKILSKGKRGGH |
| E338X | |
| 7. Cterm | KFKTSAQHALTSVSRGS |
| domain of | SLKILSKGKRG |
| E336X | |
| 8. Cterm | KFKTSAQHALTSVSRGS |
| domain of | SLKILSKGK |
| E334X | |

### REFERENCES

Throughout this application, various references describe the state of the art to which this invention pertains. The disclosures of these references are hereby incorporated by reference into the present disclosure.
1. Griffith, J. W., Sokol, C. L. & Luster, A. D. Chemokines and Chemokine Receptors: Positioning Cells for Host Defense and Immunity. Annu Rev Immunol 32, 659-702 (2014).
2. Kawai, T. & Malech, H. L. WHIM syndrome: congenital immune deficiency disease. Current Opinion in Hematology 16, 20-26 (2009).
3. Hernandez, P. A. et al. Mutations in the chemokine receptor gene CXCR4 are associated with WHIM syndrome, a combined immunodeficiency disease. Nat Genet 34, 70-74 (2003).
4. Liu, Q. et al. WHIM syndrome caused by a single amino acid substitution in the carboxy-tail of chemokine receptor CXCR4. Blood 120, 181-189 (2012).
5. Balabanian, K. et al. WHIM syndromes with different genetic anomalies are accounted for by impaired CXCR4 desensitization to CXCL12. Blood 105, 2449-2457 (2005).
6. Balabanian, K. et al. Proper desensitization of CXCR4 is required for lymphocyte development and peripheral compartmentalization in mice. Blood 119, 5722-5730 (2012).
7. Gulino, A. V. Altered leukocyte response to CXCL12 in patients with warts hypogammaglobulinemia, infections, myelokathexis (WHIM) syndrome. Blood 104, 444-452 (2004).
8. Schulz, O., Hammerschmidt, S. I., Moschovakis, G. L. & Förster, R. Chemokines and Chemokine Receptors in Lymphoid Tissue Dynamics. Annu Rev Immunol 34, annurev-immunol-041015-055649 (2014).
9. Lim, K. et al. Neutrophil trails guide influenza-specific CD8+ T cells in the airways. Science 349, aaa4352-aaa4352 (2015).
10. O'Hayre, M., Salanga, C. L., Handel, T. M. & Allen, S. J. Chemokines and cancer: migration, intracellular signalling and intercellular communication in the microenvironment. Biochem. J. 409, 635-649 (2008).
11. Gorlin, R. J. et al. WHIM syndrome, an autosomal dominant disorder: clinical, hematological, and molecular studies. Am. J. Med. Genet. 91, 368-376 (2000).
12. Guire, P. J. M., Cunningham-Rundles, C., Ochs, H. & Diaz, G. A. Oligoclonality, impaired class switch and B-cell memory responses in WHIM syndrome. Clinical Immunology 135, 412-421 (2010).
13. Badolato, R., Donadieu, J.WHIM Research Group. How I treat warts, hypogammaglobulinemia, infections, and myelokathexis syndrome. Blood 130, 2491-2498 (2017).
14. Biajoux, V. et al. Efficient Plasma Cell Differentiation and Trafficking Require Cxcr4 Desensitization. CellReports 17, 193-205 (2016).
15. Lian, J. & Luster, A. D. ScienceDirectChemokine-guided cell positioning in the lymph node orchestrates the generation of adaptive immune responses. Current Opinion in Cell Biology 36, 1-6 (2015).
16. Brinza, L. et al. Immune signatures of protectivespleen memory CD8 T cells. Sci. Rep. 1-12 (2016). doi:10.1038/srep37651
17. Ariotti, S. et al. T cell memory. Skin-resident memory CD8⁺ T cells trigger a state of tissue-wide pathogen alert. Science 346, 101-105 (2014).
18. Chaix, J. et al. Cutting Edge: CXCR4 Is Critical for CD8+Memory T Cell Homeostatic Self-Renewal but Not Rechallenge Self-Renewal. J Immunol 193, 1013-1016 (2014).
19. Mazo, I. B. et al. Bone Marrow Is a Major Reservoir and Site of Recruitment for Central Memory CD8+ T Cells. Immunity 22, 259-270 (2005).
20. Panetta, E. et al. CD8 Cell Division Maintaining Cytotoxic Memory Occurs Predominantly in the Bone Marrow. J Immunol 174, 7654-7664 (2005).
21. Sercan Alp, Ö. et al. Memory CD8+ T cells colocalize with IL-7+stromal cells in bone marrow and rest in terms of proliferation and transcription. Eur J Immunol 45, 975-987 (2015).
22. Di Rosa, F. Two Niches in the Bone Marrow: A Hypothesis onLife-long T Cell Memory. Trends Immunol 37, 503-512 (2016).
23. Alp, O. S. & Radbruch, A. The lifestyle of memory CD8(+) T cells. Nat Rev Immunol 16, 271 (2016).
24. Dotta, L., Tassone, L. & Badolato, R. Clinical and genetic features of Warts, Hypogammaglobulinemia, Infections and Myelokathexis (WHIM) syndrome. Curr. Mol. Med. 11, 317-325 (2011).
25. Mamalaki, C. et al. Positive and negative selection in transgenic mice expressing a T-cell receptor specific for influenza nucleoprotein and endogenous superantigen. Dev Immunol 3, 159-174 (1992).
26. Anderson, K. G. et al. Intravascular staining for discrimination of vascular and tissue leukocytes. Nat Protoc 9, 209-222 (2014).
27. Picelli, S. et al. Smart-seq2 for sensitive full-length transcriptome profiling in single cells. Nat. Methods 10, 1096-1098 (2013).
28. van der Woude, L. L., Gorris, M. A. J., Halilovic, A., Figdor, C. G. & de Vries, I. J. M. Migrating into the Tumor: a Roadmap for T Cells. TRENDS in CANCER 3, 797-808 (2017).
29. Feinberg, D., Paul, B. & Kang, Y. The promise of chimeric antigen receptor (CAR) T cell therapy in multiple myeloma. Cellular Immunology 103964 (2019). doi:10.1016/j.cellimm.2019.103964
30. Ghorashian, S. et al. Enhanced CAR T cell expansion and prolonged persistence in pediatric patients with ALL treated with a low-affinity CD19 CAR. Nat Med 1-23 (2019). doi:10.1038/s41591-019-0549-5
31. Khan, A. B. et al. Redirection to the bone marrow improves T cell persistence and antitumor functions. J Clin Invest 128, 2010-2024 (2018).
32. Barber, D. L. et al. Restoring function in exhausted CD8 T cells during chronic viral infection. Nature 439, 682-687 (2005).

1-8. (canceled)

9. A method of producing a T cell which expresses a chimeric antigen receptor that recognizes and binds to an antigen, comprising
transfecting or transducing, in vitro or ex vivo, a T cell that expresses a CXCR4^{Whim} mutation or that has a deletion of from 10 to 20 amino acid residues within a C-terminal domain of CXCR4, in with a vector encoding for the chimeric antigen receptor.

10. (canceled)

11. A pharmaceutical composition comprising a population of T cells that express a CXCR4^{Whim} mutation or that have a deletion of from 10 to 20 amino acid residues within a C-terminal domain of CXCR4, and a pharmaceutically acceptable carrier.

12. A method of treating an infectious disease in a subject in need thereof, comprising administering to the subject a therapeutically effective amount of T cells which express a CXCR4^{Whim} mutation or which have a deletion of 10-20 amino acid residues in a C-terminal domain of CXCR4.

13. The method according to claim 12 wherein the infectious disease is caused by a virus or a bacteria that causes lung infection.

14. A method of treating cancer in a subject in need thereof, said method comprising administering to the subject a therapeutically effective amount of T cells which express a CXCR4^{Whim} mutation or which have a deletion of 10-20 amino acid residues in a C-terminal domain of CXCR4.

15. The method according to claim 14 wherein the cancer is a hematopoietic malignancy selected from the group consisting of acute myeloid leukemia (AML), acute lymphoblastic leukemia, chronic myeloid leukemia, lymphoid leukemia, lymphoma and myelodysplastic syndrome.

16. The method of claim 12, wherein the CXCR4^{Whim} mutation is selected from the group consisting of: R334X, G336X, E343X, S341fs, S339fs342X, S338X and E343K.

17. The method of claim 12 wherein the deletion is a 10, 11, 12, 13, 14, 15, 16, 17, 18, 16, 17, 18, 19 or 20 amino acid residue deletion.

18. The method of claim 12, wherein the T cells are CD8+ T cells.

19. The method of claim 12, wherein the T cells express a chimeric antigen receptor which recognizes and binds to an antigen.

20. The method of claim 14, wherein the CXCR4^{Whim} mutation is selected from the group consisting of: R334X, G336X, E343X, S341fs, S339fs342X, S338X and E343K.

21. The method of claim 14 wherein the deletion is a 10, 11, 12, 13, 14, 15, 16, 17, 18, 16, 17, 18, 19 or 20 amino acid residue deletion.

22. The method of claim 14, wherein the T cells are CD8+ T cells.

23. The method of claim 14, wherein the T cells express a chimeric antigen receptor which recognizes and binds to an antigen.

24. The method of claim 14, wherein the T cells are administered in adoptive cell therapy.


SEQUENCE LISTING (8 sequences)

SEQ ID NO: 1 - protein, 45 aa, Homo sapiens
KFKTSAQHALTSVSRGSSLKILSKGKRGGHSSVSTESESSSFHSS

SEQ ID NO: 2 - protein, 45 aa, Homo sapiens
KFKTSAQHALTSVSRGSSLKILSKGKRGGHSSVSTKSESSSFHSS

SEQ ID NO: 3 - protein, 35 aa, Homo sapiens
KFKTSAQHALTSVSRGSSLKILSKGKRGGHSSVST

SEQ ID NO: 4 - protein, 45 aa, Homo sapiens
KFKTSAQHALTSVSRGSSLKILSKGKRGGHSSVPLSLSLQVFTPA

SEQ ID NO: 5 - protein, 34 aa, Homo sapiens
KFKTSAQHALTSVSRGSSLKILSKGKRGGHSCFH

SEQ ID NO: 6 - protein, 30 aa, Homo sapiens
KFKTSAQHALTSVSRGSSLKILSKGKRGGH

SEQ ID NO: 7 - protein, 28 aa, Homo sapiens
KFKTSAQHALTSVSRGSSLKILSKGKRG

SEQ ID NO: 8 - protein, 26 aa, Homo sapiens
KFKTSAQHALTSVSRGSSLKILSKGK